(19) United States
(12) Patent Application Publication
(10) Pub. No.: US 2022/0361950 A1
(43) Pub. Date: Nov. 17, 2022

(54) APPARATUS FOR ENDOSCOPIC SURGERY AND SYSTEM INCLUDING THE SAME

(71) Applicant: SapienMed Co., Ltd, Seoul (KR)

(72) Inventors: Seung June OH, Seoul (KR); Ji Hoon PARK, Seoul (KR); Seihoon PARK, Seoul (KR)

(73) Assignee: SapienMed Co., Ltd, Seoul (KR)

(21) Appl. No.: 17/571,504

(22) Filed: Jan. 9, 2022

Related U.S. Application Data
(60) Provisional application No. 63/188,416, filed on May 13, 2021.

(51) Int. Cl. A61B 18/24; A61B 1/307; A61B 1/00

(52) U.S. Cl. CPC A61B 18/24; A61B 1/307; A61B 1/00135; A61B 2018/00517

(57) ABSTRACT

The present disclosure provides an endoscopic surgery apparatus that includes an insertion tube having a passage formed inside, and that is to be inserted into a patient's urethra; a laser part that is installed such that it can reciprocate in the passage, and that separates a prostate adenoma from a patient's prostatic capsular surface; a hemostasis part that is installed such that it is spaced apart from the laser part and can reciprocate in the passage, and stops bleeding of a bleeding blood vessel of the patient's prostatic capsular surface; a camera part that is located in the passage and that photographs inside of the patient's prostate; and a transfer part that selectively transfers the laser part and the hemostasis part.

### 1. FIELD

The present disclosure relates to an endoscopic surgery apparatus for treatment of diseases of organs such as the prostate, bladder, ureter, kidney and the like in the field of urology, and more particularly, to an endoscopic surgery apparatus that can treat diseases of organs such as the bladder, ureter, kidney and the like through minimally invasive or noninvasive surgeries, and a system including the same.

### 2. BACKGROUND

Prostate is a male reproductive organ that surrounds the bladder neck and posterior urethra of men and that releases prostate fluid. Benign prostatic hypertrophy, which occurs due to the proliferation of prostatic adenoma, is caused by various reasons such as aging and hormonal changes. By functionally blocking the passage of the lower bladder through which urine is discharged, it causes bladder outlet obstruction, resulting in voiding symptoms such as slow stream, delayed urine and interrupted urine.

Treatments for such an enlarged prostate are divided into pharmacological treatments such as alpha blockers or androgen inhibitors, and surgical treatments of removing all or part of the prostate adenoma. Surgical treatments that use an endoscope through the urethra include transurethral resection of prostate which uses electrical energy, transurethral laser vaporization of prostate which uses laser energy, and transurethral enucleation of prostatic adenoma.

Holmium laser enucleation of prostate (HoLEP), which is a representative method for transurethral enucleation of prostatic adenoma, is in the spotlight because it can remove an enlarged prostatic adenoma from the prostatic capsular surface more completely, unlike transurethral resection of prostate and transurethral laser vaporization of prostate.

In an endoscopic surgery set used in an endoscopic surgery, independent apparatuses, basically consisting of a telescope, endoscopic working elements, and sheath, are assembled to be used in a combined form. Here, the endoscopic working elements may differ depending on the type of the surgery. Depending on the purpose of the surgery, they may be replaced by other forms of endoscopic working elements. For example, for a transurethral resection of prostate, a loop electrode for resection or hemostasis must be accommodated in the endoscopic working element. Through a working element provided with a device for conducting electricity to the tip of the electrode, as a surgeon's finger moves the loop electrode back and forth during the surgery, resection of the prostate tissue becomes possible. In the case of the Holmium laser enucleation of prostate (HoLEP), a working element that can accommodate a laser fiber is used. Using this working element, the surgeon moves the tip of the fiber back and forth, so as to remove the prostate tissue or stop the bleeding.

In a surgery, a certain energy source cannot cover everything you need in the surgery. That is, each laser has different characteristics depending on the type or wavelength of the laser. The laser with excellent vaporization function is the KTP laser, and the laser with excellent incision function is typically the Holmium laser. The transurethral resection of prostate method that uses monopolar or bipolar electrical energy shows particularly remarkable advantages in resection and hemostasis. Especially, the loop electrode that is used in the transurethral resection of prostate method enables extensive hemostasis in a short period of time, which is incomparably superior to hemostasis using laser. Most of the lasers used in urological surgeries have characteristics of hemostasis function to some extent besides specific functions. However, in cases where bleeding is severe, or when there are many blood vessels, or when such findings are expected, it is possible to reduce the surgery time by preemptively performing hemostasis to smooth the surgical process.

There are cases where the purpose of the surgery changes in detail during a specific surgery. For example, there are cases where hemostasis is required during a tissue resection, that is, when bleeding needs to be stopped using electrical energy during a surgery using laser. In this case, it is necessary to stop the surgery, disassemble the endoscopy, replace the endoscopic working element to another and reassemble the endoscopy, then connect electricity or laser, and resume the surgery.

FIGS. 1A to 1D are views illustrating a prior art Holmium laser prostatectomy surgery process. Referring to FIGS. 1A to 1D, a surgeon inserts a working element 20 into a patient's body through a urethra 13, and irradiates laser 21 from the laser fiber accommodated in the endoscopic working element 20, thereby separating the prostatic adenoma 11 from the prostatic capsular surface 12. Then, the surgeon pushes the prostatic adenoma 11 separated from the prostatic capsular surface 12 inside the bladder 10, so that the separated prostatic adenoma 11 can be collected inside the bladder 10. Since the collected prostatic adenoma 11 is too big to pass through the urethra, it is morcellated into pieces in the bladder 10 by an equipment such as a morcellator, and then discharged outside.

Meanwhile, when separating the prostatic adenoma from the prostatic capsular surface using Holmium laser, hemostasis of the bleeding blood vessel must be smoothly performed. Otherwise, because of the bleeding, it may become difficult to secure the surgical field of view and proceed with the surgery smoothly. Further, when a perforation is made on the prostatic capsular surface on the outside of the prostatic adenoma during a surgery caused by difficulty of securing the surgical field of view due to bleeding, massive bleeding might be caused from the large venous blood vessels 14 on the outside of the capsular surface, which may lead to a very dangerous situation.

Therefore, upon separating the prostatic adenoma 11 from the prostatic capsular surface 12, in many cases, the surgeon has to additionally perform a process of stopping bleeding of a blood vessel in the prostatic capsular surface 12 using an endoscopic working element 20 having a bipolar electrode 22 and the like for hemostasis. As mentioned above, for this purpose, during the surgery, the surgeon has to reassemble the endoscopic working element 20 for hemostasis, and then insert the assembled endoscopic working element 20 for hemostasis into the patient's urethra.

Not only in Holmium laser enucleation of prostate (HoLEP), but also in bladder tumor resection using laser, internal urethrotomy, bladder neck incision, and endopyelotomy, in many cases, hemostasis is necessary after performing an incision and resection using laser. Also in these cases, after using laser, it is often necessary to additionally perform the process of stopping the bleeding of a blood vessel on the prostatic capsular surface 12 using a new endoscopic working element 20 for hemostasis.

As such, reassembling a surgical equipment is a cumbersome process, as it causes distraction during surgery, time delays such as temporary suspension of surgery, and complexity of changing settings. This may place a burden on the surgeon who must pay close attention to the patient's anatomy and the patient's condition during surgery, and is not desirable either from the perspective of the patient undergoing surgery. Accordingly, there is a need for an endoscopic working element that does not require a reassembly of separate surgical equipment during a surgical process.

### SUMMARY

Therefore, in order to resolve the above-mentioned problems, embodiments of the present disclosure intend to provide an endoscopic surgery apparatus that is mechanically supplemented to enable a surgeon to focus only on the surgery without having to reassemble a separate surgical equipment in the process of performing an endoscopic surgery, and a system including the same.

Further, embodiments of the present disclosure intend to provide an endoscopic surgery apparatus that enables medical staff to control movements of a laser part and a hemostasis part necessary for prostatic adenoma separation and prostatic capsular surface hemostasis with ease and stability, and a system including the same.

Tasks of the present application are not limited to the tasks mentioned above, and any nonmentioned tasks will be clearly understood by one skilled in the art from the following description.

An endoscopic surgery apparatus according to an embodiment of the preset disclosure includes an insertion tube having a passage formed inside, and that is to be inserted into a patient's urethra; a laser part that is installed such that it can reciprocate in the passage, and that separates a prostate adenoma from a patient's prostatic capsular surface; a hemostasis part that is installed such that it is spaced apart from the laser part and can reciprocate in the passage, and stops bleeding of a bleeding blood vessel of the patient's prostatic capsular surface; a camera part that is located in the passage and that photographs inside of the patient's prostate; and a transfer part that selectively transfers the laser part and the hemostasis part.

The camera part may be disposed at a center of the passage, the hemostasis part may be disposed to be upwardly spaced apart from the camera part, and the laser part may be disposed to be downwardly spaced apart from the camera part.

The endoscopic surgery apparatus may further include a guider that guides the laser part, the hemostasis part and the camera part to be spaced apart from one another inside the passage.

The endoscopic surgery apparatus may further include a blocking block that is installed at one side of the insertion tube, and where the laser part and the hemostasis part are each slided, and where the camera part is immobilized.

The transfer part may include a first block that immobilizes one side end of the laser part, and that reciprocates the laser part in the passage; a second block that is located adjacent to the first block, and that immobilizes one side end of the hemostasis part, and that reciprocates the hemostasis part in the passage; and a third block that is selectively connected to the first block and the second block, and the third block may selectively transfer the first block and the second block.

The endoscopic surgery apparatus may further include a hinge having one side connected to the blocking block, and the other side connected to the third block.

A first handle may be immobilized to the blocking block, and a second handle may be installed in the third block, and thus when the second handle is moved towards the first handle, the third block may move towards one side of the insertion tube.

An endoscopic surgery system according to another embodiment of the present disclosure includes an endoscopic surgery apparatus; a display that is connected to the endoscopic surgery apparatus, to display an image photographed in the endoscopic surgery apparatus; and a controller that controls the endoscopic surgery apparatus and the display, wherein the endoscopic surgery apparatus may include an insertion tube having a passage formed inside, and that is to be inserted into a patient's urethra; a laser part that is installed such that it can reciprocate in the passage, and that separates a prostate adenoma from a patient's prostatic capsular surface; a hemostasis part that is installed such that it is spaced apart from the laser part and can reciprocate in the passage, and stops bleeding of a bleeding blood vessel of the patient's prostatic capsular surface; a camera part that is located in the passage and that photographs inside of the patient's prostate; and a transfer part that selectively transfers the laser part and the hemostasis part.

An endoscopic surgery apparatus according to an embodiment of the present disclosure can perform hemostasis on a bleeding blood vessel within a short period of time upon detachment of prostatic adenoma, and thus provides an effect of sufficiently securing the surgical field of view.

Further, when using the endoscopic surgery apparatus according to an embodiment of the present disclosure, there is no need for a process of reassembling the surgical equipment during the surgery, and thus provides and effect of reducing the surgery time.

The effects of the present applications are not limited to those mentioned above, and any unmentioned effects will be clearly understood by those skilled in the art from the following description.

### BRIEF DESCRIPTION OF THE DRAWINGS

FIGS. 1A to 1D are illustrations of a prior art Holmium laser prostatectomy surgery process;

FIG. 2 is a perspective view of an endoscopic surgery apparatus according to an embodiment of the present disclosure;

FIG. 3 is a perspective view with an insertion tube of the endoscopic surgery apparatus of FIG. 2 removed;

FIG. 4 is a view for describing a guider that guides a laser part, a hemostasis part and a camera part of the endoscopic surgery apparatus of FIG. 2;

FIG. 5 is a cross-sectional perspective view cut along the insertion tube portion of the endoscopic surgery apparatus of FIG. 2;

FIG. 6 is an illustration when seen from the front of the cut portion of FIG. 5;

FIG. 7 is a view for describing a process where the laser part of the endoscopic surgery apparatus of FIG. 2 moves in the insertion tube;

FIG. 8 is a view for describing a process where the hemostasis part of the endoscopic surgery apparatus of FIG. 2 moves in the insertion tube;

FIGS. 9A and 9B are views for describing a transfer part of the endoscopic surgery apparatus of FIG. 2;

FIGS. 10 to 14 are views for describing the transfer part according to another embodiment of the present disclosure; and

FIG. 15 is a schematic view of an endoscopic surgery system according to another embodiment of the present disclosure.

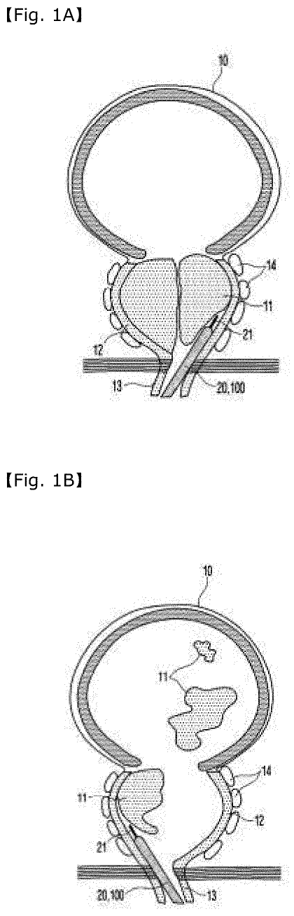

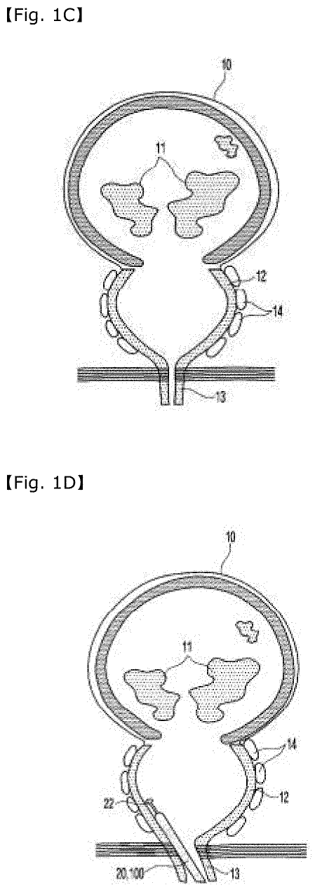

### DETAILED DESCRIPTION

Hereinbelow, embodiments of the present disclosure will be described in detail with reference to the accompanying drawings. However, those skilled in the art should easily understand that the accompanying drawings are only described to more easily disclose the contents of the present disclosure, and that the scope of the present disclosure is not limited to the scope of the accompanying drawings.

Further, terms used in the detailed description of the present disclosure and in the claims set are merely for describing specific embodiments, and not for limiting the present disclosure. Singular expressions include plural expressions unless the context clearly indicates otherwise.

In the detailed description of the present disclosure and the claims set, it should be understood that terms such as “include/comprise” or “have” are intended to designate that features, numbers, steps, operations, components, parts or combinations thereof disclosed in the specification are present, and not to exclude the possibility of presence or addition of one or more other features, numbers, operations, components, parts or combinations thereof.

Moreover, the present disclosure covers all possible combinations of the embodiments indicated in the detailed description. It should be understood that various embodiments of the present disclosure are different from one another, but need not be mutually exclusive. For example, specific shapes, structures or features disclosed herein may be implemented in other embodiments without departing from the spirit and scope of the present disclosure in relation to one embodiment. Further, it should be understood that the location or arrangement of individual components within each disclosed embodiment may be changed without departing from the spirit and scope of the present disclosure. Therefore, the detailed description to be described later is not intended to be taken in a limited sense, and the scope of the present disclosure, if properly described, is limited only by the accompanying claims, along with all scopes equivalent to those claimed by the claims. Like reference numerals in the drawings refer to the same or similar functions throughout the various aspects.

Hereinafter, an endoscopic surgery apparatus and a system including thereof according to an embodiment of the present disclosure will be described with reference to the accompanying drawings.

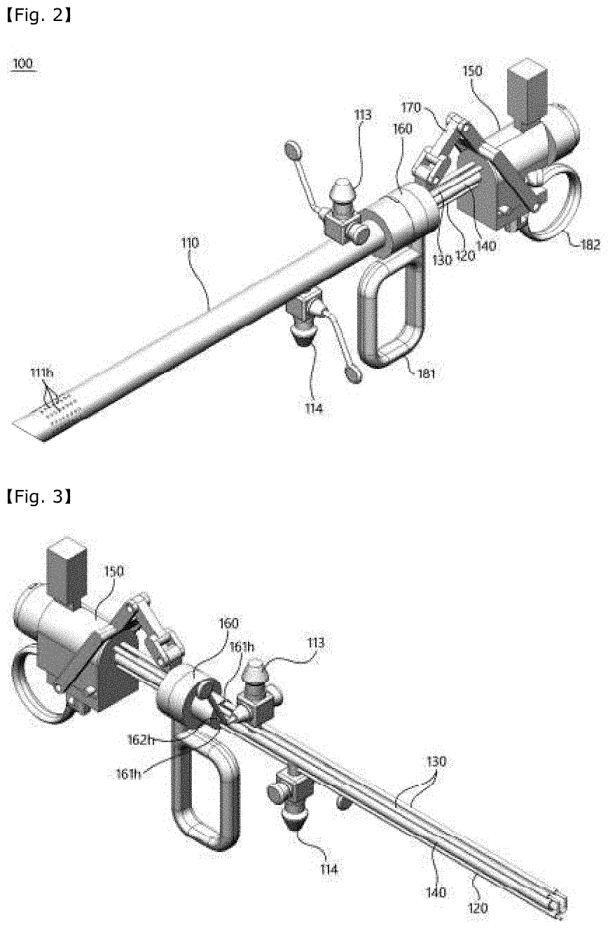

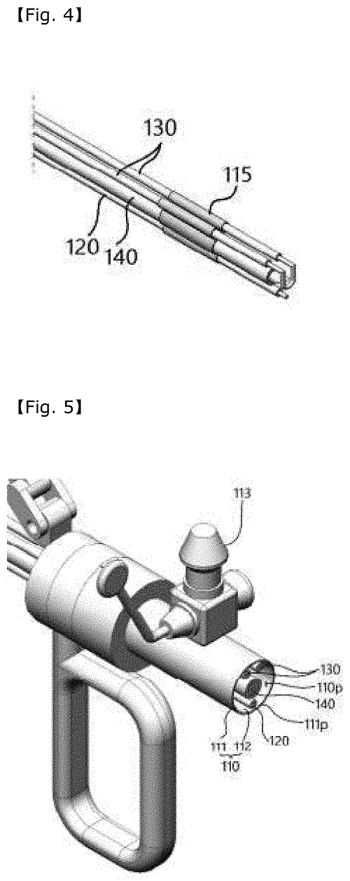

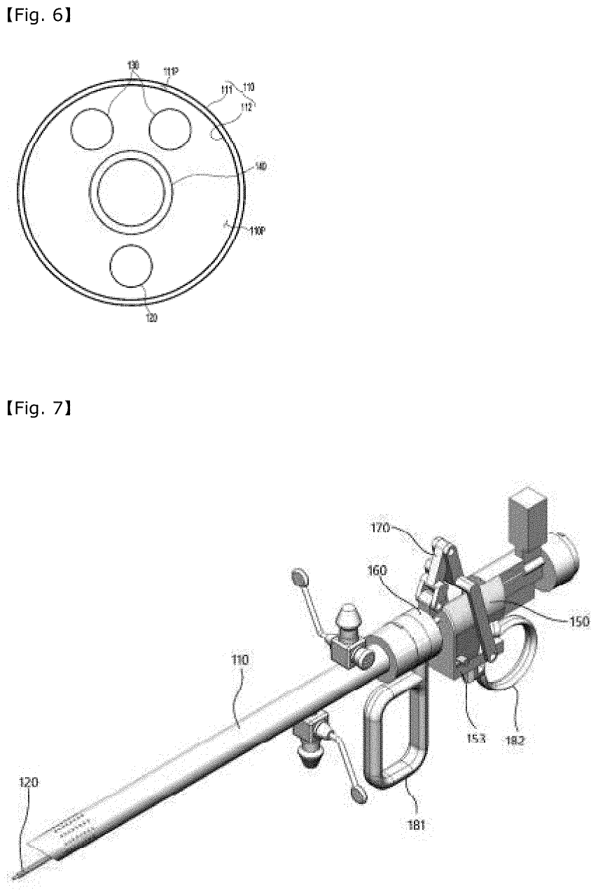

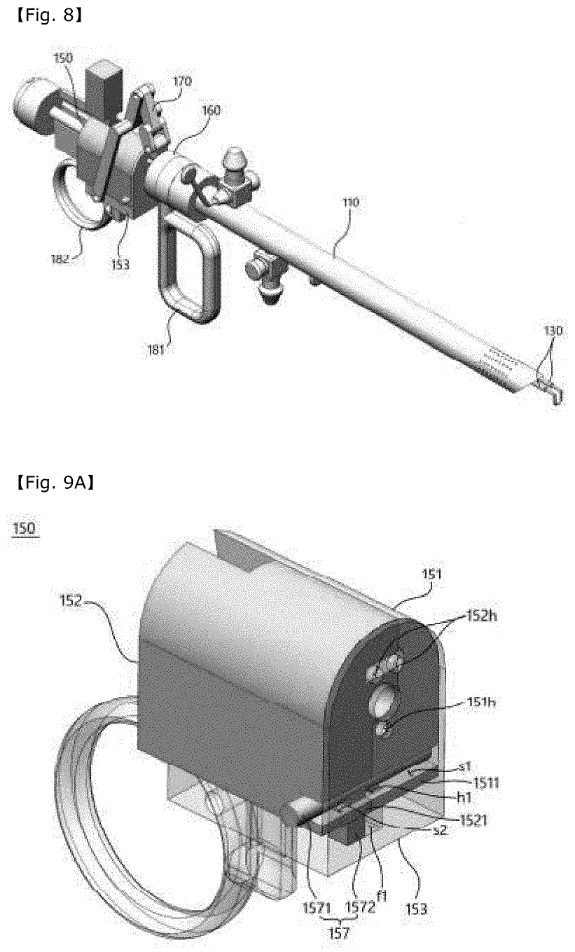

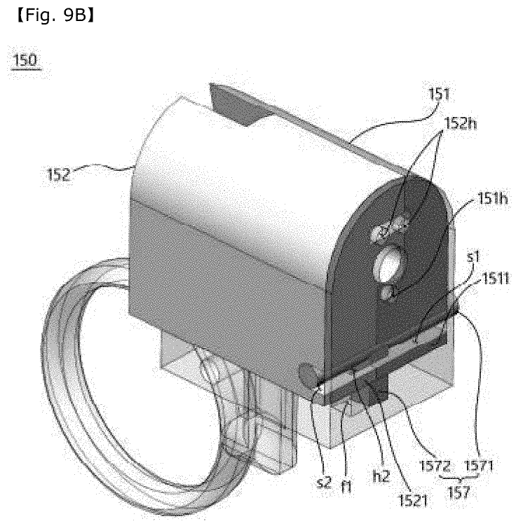

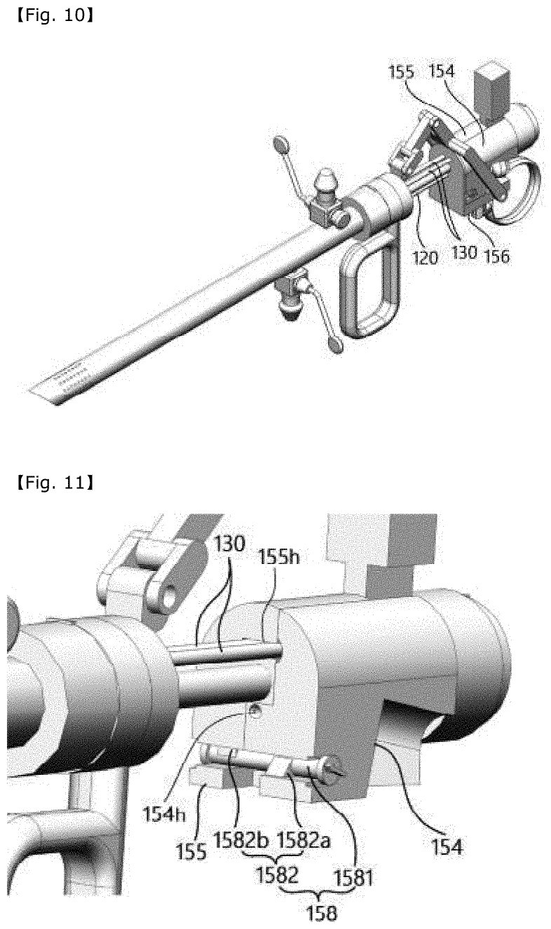

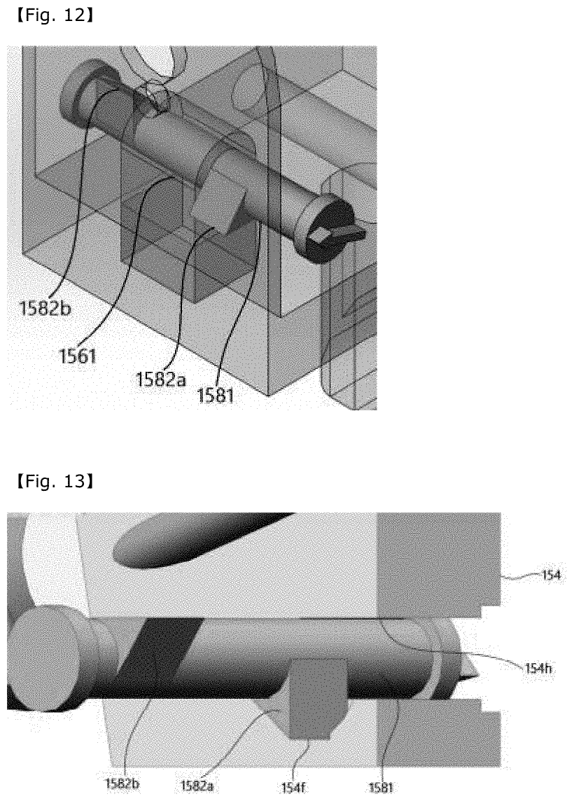

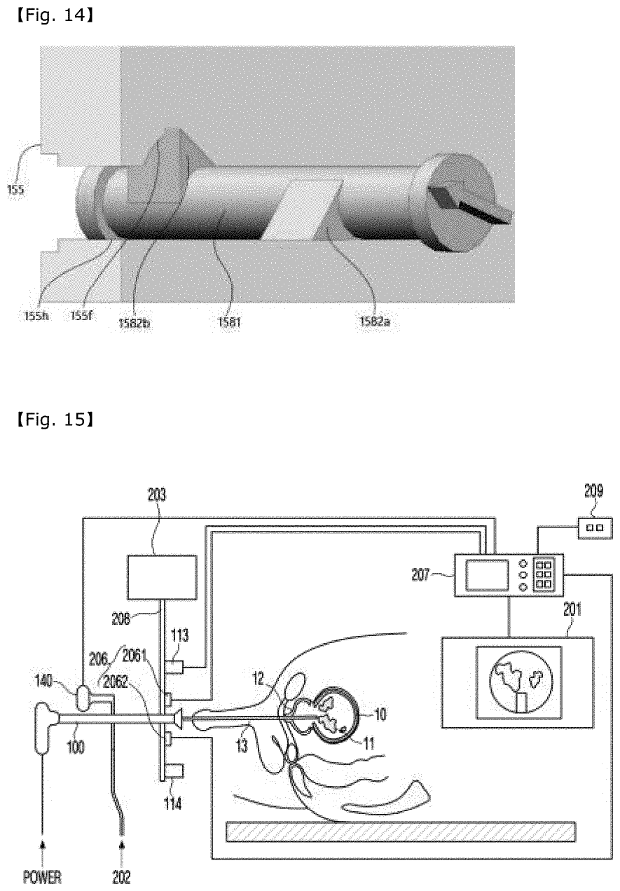

FIG. 2 is a perspective view of an endoscopic surgery apparatus according to an embodiment of the present disclosure, FIG. 3 is a perspective view with an insertion tube of the endoscopic surgery apparatus of FIG. 2 removed, FIG. 4 is a view for describing a guider that guides a laser part, a hemostasis part and a camera part of the endoscopic surgery apparatus of FIG. 2, FIG. 5 is a cross-sectional perspective view cut along the insertion tube portion of the endoscopic surgery apparatus of FIG. 2, FIG. 6 is an illustration when seen from the front of the cut portion of FIG. 5, FIG. 7 is a view for describing a process where the laser part of the endoscopic surgery apparatus of FIG. 2 moves in the insertion tube, FIG. 8 is a view for describing a process where the hemostasis part of the endoscopic surgery apparatus of FIG. 2 moves in the insertion tube, FIGS. 9A and 9B are views for describing a transfer part of the endoscopic surgery apparatus of FIG. 2, FIGS. 10 to 14 are views for describing the transfer part according to another embodiment of the present disclosure, and FIG. 15 is a schematic view of an endoscopic surgery system according to another embodiment of the present disclosure.

Referring to FIGS. 2 to 9B, an endoscopic surgery apparatus according to an embodiment of the present disclosure includes an insertion tube 110, a laser part 120, a hemostasis part 130, a camera part 140 and a transfer part 150.

The insertion tube 110 is inserted into a patient's urethra, and a passage 110p is formed inside the insertion tube 110.

Referring to FIG. 6, in the passage 110p, the laser part 120, the hemostasis part 130 and the camera part 140 are located. More specifically, as in FIG. 6 that illustrates a cross-section of the insertion tube 110 that surrounds the laser part 120, the hemostasis part 130 and the camera part 140, the camera part 140 is disclosed at a center of the insertion tube 110, and the hemostasis part 130 and the laser part 120 may be disposed above and below the camera part 140, respectively.

The hemostasis part 130 and the laser part 120 may be disposed at opposite positions to each other, and the hemostasis part 130 and the laser part 120 may be variously disposed based on the center of the insertion tube, but it is desirable that the camera part 140 is disposed at the center of the insertion tube 110 so that the surgeon can have a wide viewing angle through the camera part 140 when the hemostasis part 130 and the laser part 120 operate. Through the passage 110p, fluid such as water and the like may move.

Tips (end portions) of the laser part 120 and the hemostasis part 130 may each be exposed to outside at an end portion of the insertion tube 110.

The insertion tube 110 may include an outer sheath 111 and an inner sheath 112.

The inner sheath 112 is located inside the outer sheath 111, and an outer surface of the inner sheath 112 is disposed to be spaced apart from an inner surface of the outer sheath 111.

The outer surface of the inner sheath 112 may be disposed to be spaced apart from the inner surface of the outer sheath 111, thereby forming a discharge passage 111p through which the fluid can flow.

In the inner sheath 112, the passage 110p is formed, and through the passage 110p, the fluid from outside can be introduced inside the patient's prostatic capsular surface or bladder.

At a tip portion of the outer sheath 111, a suction hole 111h may be formed.

The suction hole 111h spatially communicates with the discharge passage 111p, and the fluid inside the patient's prostatic capsular surface 12 or inside the bladder 10 may pass through the suction hole 111h, and be discharged outside through the discharge passage 111p.

In the insertion tube 110, a first valve 113 and a second valve 114 may be installed.

The first valve 113 may be connected to the passage 110p, and the second valve 114 may be connected to the discharge passage 111p.

When the first valve 113 is opened, the fluid from outside may pass through the first valve 113, and then be introduced inside the prostatic capsular surface 12 or inside the bladder 10 through the passage 110p, and when the second valve 114 is opened, the fluid inside the prostatic capsular surface 12 or the bladder 10 may be suctioned into the suction hole 111h, to pass through the discharge passage 111p and the second valve 114, and discharged outside.

The laser part 120 is installed inside the passage 110p such that it can reciprocate in the passage 110p.

In the laser part 120, the medium for irradiating laser is made of optical fiber, but is not limited thereto, and any medium capable of irradiating laser may be used. Such a laser part 120 may separate the prostate adenoma 11 from the prostatic capsular surface 12 with laser beam being emitted from the optical fiber.

Specifically, when separating the prostate adenoma 11 from the prostatic capsular surface 12, the tip of the laser part 120 may be exposed outside the end portion of the insertion tube 110, and irradiate laser between the prostatic capsular surface 12 and the prostate adenoma 11.

The hemostasis part 130 may be installed to be spaced apart from the laser part 120, and to reciprocate in the passage 110p, and may stop the bleeding of a bleeding blood vessel of the prostatic capsular surface 12 when separating the prostate adenoma 11 from the prostatic capsular surface 12.

The hemostasis part 130 may be any type of electrode such as BOVIE and the like as long as it can stop the bleeding of a bleeding site. As the electrode, both a bipolar electrode or a unipolar electrode may be used.

Specifically, the tip of the hemostasis part 130 may apply heat on the bleeding site of the prostatic capsular surface 12, that occurred when separating the prostate adenoma 11 from the prostatic capsular surface 12, to stop the bleeding.

In the process of separating the prostate adenoma 11 from the prostatic capsular surface 12, the camera part 140 may photograph images of the prostatic capsular surface 12, the prostate adenoma 11, area surrounding the tip of the laser part 120, and the tip portion of the hemostasis part 130.

In the passage 110p of the insertion tube 110, a guider 115 may be installed.

Specifically, the guider 115 may be located in the inner sheath 112 and immobilized to the camera part 140, and a guide hole may be formed in which the laser part 120 and the hemostasis part 130 can move.

Therefore, in the passage 110, the guider 115 may guide the laser part 120 and the hemostasis part 130 so that the laser part 120 and the hemostasis part 130 can reciprocate in a length direction in a state where the laser part 120 and the hemostasis part 130 are spaced apart from each other.

The transfer part 150 may be connected to one side end of the insertion tube 110, and selectively move either one of the laser part 120 and the hemostasis part 130 in the insertion tube 110, and expose the tip of the laser part 120 and the hemostasis part 130 to outside of the end portion of the insertion tube 110.

Specifically, when it is necessary to separate the prostate adenoma 111 from the prostatic capsular surface 12, the transfer part 150 may move the laser part 120 such that the tip of the laser part 120 is exposed outside of the end portion of the insertion tube 110.

Further, when it is necessary to stop the bleeding of a bleeding site of the prostatic capsular surface 12, the transfer part 150 may move the hemostasis part 130 such that the tip of the hemostasis part 130 is exposed outside at the end portion of the insertion tube 110.

The endoscopic surgery apparatus 100 may further include a blocking block 160.

The blocking block 160 may be installed at one side of the insertion tube 110, to block one side of the insertion tube 110.

The laser part 120, the hemostasis part 130 and the camera part 140 may each be slided against the blocking block 160 and be immobilized. For this purpose, the blocking block 160 may have a sliding hole 161h which the laser part 120 and the hemostasis part 130 may each pass through and be slided, and an immobilizing hole 162h which the camera part 140 may pass through and be immobilized.

The transfer part 150 may include a first block 151, a second block 152 and a third block 153.

The first block 151 and the second block 152 may be slided against each other, and only when the first block 151 and the second block 152 are each mechanically connected with the third block 153, they may be moved in a front and back direction by the third block 153.

To the first block 151, one side end of the laser part 120 is immobilized, and the first block 151 may reciprocate the laser part 120 in the passage 110p.

Specifically, referring to FIGS. 7 and 9B, when one side end of the laser part 120 is inserted into the hole 151h of the first block 151 and is electrically coupled to a power terminal (not illustrated) inside the first block 151, and the first block 151 moves in a direction closer to the insertion tube 110, the laser part 120 moves forward to a distal end direction of the insertion tube in the sliding hole 161h and the passage 110p. Accordingly, the tip of the laser part 120 may be exposed outside of the end portion of the insertion tube 110.

When the first block 151 moves in a direction away from the insertion tube 110, the laser part 120 moves backward in a proximal end direction of the insertion tube in the sliding hole 161h and the passage 110p. Accordingly, the tip of the laser part 120 may return back to inside of the passage 110p.

The second block 152 is located adjacent to the first block 151.

To the second block 152, one side end of the hemostasis part 130 is immobilized, and the second block 152 may reciprocate the hemostasis part 130 in the passage 110p.

Specifically, referring to FIGS. 7 and 9A, when one side end of the hemostasis part 130 is inserted into the hole 152h of the second block 152, and is electrically coupled to a power terminal (not illustrated) that is inside the second block 152, and the second block 152 moves in a direction closer to the insertion tube 110, the hemostasis part 130 moves forward in a distal end direction of the insertion tube 110 in the sliding hole 161h and the passage 110p. Accordingly, the tip of the hemostasis part 130 may be exposed to outside of the end of the insertion tube 110.

When the second block 152 moves in a direction away from the insertion tube 110, the hemostasis part 130 moves backward in a proximal end direction of the insertion tube in the sliding hole 161h and the passage 110p. Accordingly, the tip of the hemostasis part 130 may return back to the inside of the passage 110p.

The third block 153 may be selectively connected to the first block 151 and the second block 152, and selectively transfer the first block 151 and the second block 152.

Specifically, when the third block 153 is connected with the first block 151, the third block 153 may move the first block 151 in a back and forth direction, and when the third block 153 is connected with the second block 152, the third block 153 may move the second block 152 in a back and forth direction.

The transfer part 150 may further include a first switch 157 that is a mechanical structure.

The first switch 157 may selectively connect the first block 151 and the second block 152 to the third block 153.

The first switch 157 may include a push rod 1571 and a stumbling piece 1572. The push rod 1571 and the stumbling piece 1572 of the first switch 157 may form an integral body, and may be made of a material having a greater strength than those of the materials of the first block 151, the second block 152 and the third block 153, in order to reduce the wear characteristics caused by multiple movements of the surgeon. The stumbling piece 1572 may be connected to one end of the push rod 1571 vertically to a longitudinal direction of the push rod 1571.

The push rod 1571 may be inserted into sliding grooves s1, s2 each formed in left and right direction in each of the first block 151 and the second block 152.

Hereinafter, the left and right direction of the endoscopic surgery apparatus 100 is based on the direction in which the surgeon sees the patient during the procedure using the endoscopic surgery apparatus 100.

The push rod 1571 may move in left and right direction in the sliding grooves s1, s2.

An upper surface of the third block 153 is provided with an immobilizing groove f1 in which a lower portion of the stumbling piece 1572 may be inserted.

The stumbling piece 1572 may move in left and right direction depending on the movement direction of the push rod 1571 in the immobilizing groove f1.

On a lower end of the first block 151, a first slider guider 1511 may be extended so as to provide the first sliding groove s1.

On the first slider guider 1511, a first stumbling groove h1 that is connected with the first sliding groove s1 may be provided.

On a lower end of the second block 152, a second slider guider 1521 may be extended so as to provide a second sliding groove s2.

On the second slider guider 1521, a second stumbling groove h2 that is connected with the second sliding groove s2 may be provided.

The first stumbling groove h1 is connected to a left side of the immobilizing groove f1, and the second stumbling groove h2 is connected with a right side of the immobilizing groove f1.

When the push rod 1571 moves to the left side of the immobilizing groove f1, the stumbling piece 1572 is inserted into the first stumbling groove h1, and when the push rod 1571 moves to the right side of the immobilizing groove f1, the stumbling piece 1572 is inserted into the second stumbling groove h2.

When the stumbling piece 1572 is inserted into the first stumbling groove h1, by the stumbling piece 1572, the first block 151 is connected with the third block 153, and the third block 153 may move the first block 152 forward or backward.

When the stumbling piece 1572 is inserted into the second stumbling groove h2, by the push rod 1571, the second block 152 is connected with the third block 153, and the third block 153 may move the second block 152 forward or backward.

The endoscopic surgery apparatus 100 may further include a hinge 170.

One side of the hinge 170 may be connected to the blocking block 160, and the other side of the hinge 170 may be connected to the third block 153, to guide the movement of the third block 153.

On the blocking block 160, a first handle 181 may be immobilized, and on the third block 153, a second handle 182 may be installed.

When the surgeon moves the second handle 182 towards the first handle 181, the third block 153 moves closer to the insertion tube 110, and when the second handle 182 is moved away from the first handle 181, the third block 153 moves away from the insertion tube 110.

Referring to FIGS. 10 to 14, according to another embodiment of the present disclosure, the transfer part 15 may include a fourth block 154, a fifth block 155 and a sixth block 156.

The fourth block 154 and the fifth block 155 may be slided against each other, and only when the fourth block 154 and the fifth block 155 are each mechanically connected with the sixth block 156, the fourth block 154 and the fifth block 155 may be moved in back and forth direction by the sixth block 156.

On the fourth block 154, one side end of the laser part 120 is immobilized, and the fourth block 154 may reciprocate the laser part 120 in the passage 110p.

Specifically, referring to FIGS. 10 and 11, when one side end of the laser part 120 is inserted into the hole 154h of the fourth block 154 and is electrically coupled with the power terminal (not illustrated) that is inside the fourth block 154, and the fourth block 154 moves in a closer direction to the insertion tube 110, the laser part 120 moves forward in a distal end direction of the insertion tube in the sliding hole 161h and the passage 110p. Accordingly, the tip of the laser part 120 may be exposed to outside of the end portion of the insertion tube 110.

When the fourth block 154 moves in a direction away from the insertion tube 110, the laser part 120 moves backward in a proximal end direction of the insertion tube in the sliding hole 161h and the passage 110p. Accordingly, the tip of the laser part 120 may be returned back to the inside of the passage 110p.

The fifth block 155 is located adjacent to the fourth block 154.

On the fifth block 155, one side end of the hemostasis part 130 is immobilized, and the fifth block 155 may reciprocate the hemostasis part 130 in the passage 110p.

Specifically, when one side end of the hemostasis part 130 is inserted into the hole 155h of the fifth block 155 and is electrically coupled with the power terminal (not illustrated) that is inside the fifth block 155, and the fifth block 155 moves in a closer direction to the insertion tube 110, the hemostasis part 130 moves forward in a distal end direction of the insertion tube 110 in the sliding hole 161h and the passage 110p. Accordingly, the tip of the hemostasis part 130 may be exposed to outside of the end portion of the insertion tube 110.

When the fifth block 155 moves in a direction away from the insertion tube 110, the hemostasis part 130 moves backward in a proximal end direction of the insertion tube in the sliding hole 161h and the passage 110p. Accordingly, the tip of the hemostasis part 130 may return back to the inside of the passage 110p.

The sixth block 156 may be selectively connected to the fourth block 154 and the fifth block 155, and selectively transfer the fourth block 154 and the fifth block 155.

Specifically, when the sixth block 156 is connected with the fourth block 154, the sixth block 156 may move the fourth block 154 in a back and forth direction, and when the sixth block 156 is connected with the fifth block 155, the six block 156 may move the fifth block 155 in a back and forth direction.

The transfer part 150 may further include a second switch 158 which is a mechanical structure.

The second switch 158 may selectively connect the sixth block 156 to the fourth block 154 and the fifth block 155.

The second switch 158 may include a rotating rod 1581 and a stumbling projection 1582.

The stumbling projection 1582 may be formed to protrude from an outer surface of the rotating rod 1581.

The stumbling projection 1582 may include a first stumbling projection 1582a and a second stumbling projection 1582b, that are disposed to be spaced apart from each other.

Specifically, the sixth block 156 may be provided with an axis block 1561 having a rotating hole on which the rotating rod 1581 may rotate. The rotating rod 1581 is inserted into the axis block 1561, and the first stumbling projection 1582a and the second stumbling projection 1582b are connected to the rotating rod 1581 such that they are positioned at both sides of the axis block 1561.

The fourth block 154 is provided with a first rotating groove 154h on which the rotating rod 1581 may rotate based on an axial direction, and on a lower portion of the first rotating groove 154h, a first insertion groove 154f may be formed, that is where the first stumbling projection 1582a may be inserted.

Further, the fifth block 155 is provided with a second rotating groove 155h on which the rotating rod 1581 may rotate based on the axial direction, and on an upper portion of the second rotating groove 155h, a second insertion groove 155f may be formed, that is where the second stumbling projection 1582b may be inserted.

When the rotating rod 1581 rotates in a first direction, the first stumbling projection 1582a is inserted into the first insertion groove 154f, wherein the second stumbling projection 1582b is deviated from the second insertion groove 155f. In addition, when the rotating rod 1581 rotates in a second direction, that is the opposite direction to the first direction, the first stumbling projection 1582a is deviated from the first insertion groove 154f, and the second stumbling projection 1582a is inserted into the second insertion groove 155f.

When the first stumbling projection 1582a is inserted into the first insertion groove 154f, the sixth block 156 may be connected with the fourth block 154 and transfer the fourth block 154 in a back and forth direction.

When the second stumbling projection 1582b is inserted into the second insertion groove 155f, the sixth block 156 may be connected with the fifth block 155 and transfer the fifth block 155 in a back and forth direction.

Referring to FIG. 15, an endoscopic surgery system according to another embodiment of the present disclosure 200 includes a display 201, a light source 202, an irrigation fluid container 203, an optical cable 204, a laser supply 205, a sensor 206, a controller 207 and the endoscopic apparatus 100.

The display 201 may be connected with the camera part 140, and display an image photographed in the camera part 140, through a screen.

The irrigation fluid container 203 may be connected with the first valve 113 through a tube 208.

When the first valve 113 is opened, the fluid stored in the irrigation fluid container 203 may be introduced into the insertion tube 110 of the endoscopic surgery apparatus 100 through the first valve 113.

The optical cable 204 is connected with the camera part 140.

Light that is introduced through the optical cable 204 may illuminate the light necessary for photographing from the tip portion of the camera part 140 to the prostatic capsular surface 12, prostate adenoma 11, area surrounding the tip of the laser part 120 and the tip portion of the hemostasis part 130 and the like.

The laser supply 205 may be connected with the laser part 120, and may provide laser to the laser fiber of the laser part 120.

The sensor 206 may include a first sensor 2061 and a second sensor 2062.

The first sensor 2061 may be installed adjacent to the first valve 113, and measure the pressure of the fluid going into the bladder.

The second sensor 2062 may be installed adjacent to the second valve 114 and measure the pressure of the fluid coming out of the bladder.

The controller 207 may be connected to each of the display 201, light source 202, optical cable 204, laser supply 205 and sensor 206, and control the display 201, light source 202, optical cable 204, laser supply 205 and sensor 206.

The controller 207 may generate a video image clip file from an image photographed in the camera 140, and the video image clip file may be part of an image data corresponding to the section set by the user or the surgeon. The controller 207 may associate a memo regarding the video image clip file and store it in a memory 209. The memo may be the surgeon's memo about the video clip file, but there is no limitation thereto. For example, the memo may include a main memo about the name of surgery and a sub memo about a preview.

The embodiments according to the present disclosure have been described above, and the fact that the present disclosure can be embodied in other specific forms without departing from its spirit or scope in addition to the above-described embodiments is obvious for those skilled in the art. Therefore, the above-mentioned embodiments are to be regarded as illustrative rather than restrictive, and accordingly, the present disclosure is not limited to the above description, but may be modified within the scope of the accompanying claims and their equivalents.

What is claimed is:

1. An endoscopic surgery apparatus comprising:
an insertion tube having a passage formed inside, and that is to be inserted into a patient's urethra;
a laser part that is installed such that it can reciprocate in the passage, and that separates a prostate adenoma from a patient's prostatic capsular surface;
a hemostasis part that is installed such that it is spaced apart from the laser part and can reciprocate in the passage, and stops bleeding of a bleeding blood vessel of the patient's prostatic capsular surface;
a camera part that is located in the passage and that photographs inside of the patient's prostate; and
a transfer part that selectively transfers the laser part and the hemostasis part.

2. The endoscopic surgery apparatus according to claim 1,
wherein the camera part is disposed at a center of the passage,
the hemostasis part is disposed to be upwardly spaced apart from the camera part, and
the laser part is disposed to be downwardly spaced apart from the camera part.

3. The endoscopic surgery apparatus according to claim 2,
further comprising a guider that guides the laser part, the hemostasis part and the camera part to be spaced apart from one another inside the passage.

4. The endoscopic surgery apparatus according to claim 1,
further comprising a blocking block that is installed at one side of the insertion tube, and where the laser part and the hemostasis part are each slided, and where the camera part is immobilized.

5. The endoscopic surgery apparatus according to claim 4,
wherein the transfer part comprises a first block that immobilizes one side end of the laser part, and that reciprocates the laser part in the passage;
a second block that is located adjacent to the first block, and that immobilizes one side end of the hemostasis part, and that reciprocates the hemostasis part in the passage; and
a third block that is selectively connected to the first block and the second block, and
the third block selectively transfers the first block and the second block.

6. The endoscopic surgery apparatus according to claim 5,
further comprising a hinge having one side connected to the blocking block, and the other side connected to the third block.

7. The endoscopic surgery apparatus according to claim 7,
wherein a first handle is immobilized to the blocking block, and a second handle is installed in the third block, and thus when the second handle is moved towards the first handle, the third block moves towards one side of the insertion tube.

8. An endoscopic surgery system comprising:
an endoscopic surgery apparatus;
a display that is connected to the endoscopic surgery apparatus, to display an image photographed in the endoscopic surgery apparatus; and
and a controller that controls the endoscopic surgery apparatus and the display,
wherein the endoscopic surgery apparatus comprises an insertion tube having a passage formed inside, and that is to be inserted into a patient's urethra;
a laser part that is installed such that it can reciprocate in the passage, and that separates a prostate adenoma from a patient's prostatic capsular surface;
a hemostasis part that is installed such that it is spaced apart from the laser part and can reciprocate in the passage, and stops bleeding of a bleeding blood vessel of the patient's prostatic capsular surface;
a camera part that is located in the passage and that photographs inside of the patient's prostate; and
a transfer part that selectively transfers the laser part and the hemostasis part.
